(19) United States
(12) Patent Application Publication
(10) Pub. No.: US 2017/0251967 A1
(43) Pub. Date: Sep. 7, 2017

(54) SYSTEM, APPARATUS AND METHOD FOR INDIVIDUALIZED STRESS MANAGEMENT

(71) Applicant: FlourishiQ Inc., Kitchener (CA)

(72) Inventor: Hardy PREMSUKH, Kitchener (CA)

(21) Appl. No.: 15/060,826

(22) Filed: Mar. 4, 2016

(51) Int. Cl. A61B 5/16; A61B 5/024; A61B 5/00

(52) U.S. Cl. CPC A61B 5/165; A61B 5/0004; A61B 5/742; A61B 5/02405; A61B 5/683

(57) ABSTRACT

System, apparatus and method for the management of stress of one or more users. One such system comprises providing an indication to a user based correlating physiological data, allostatic load data and psychological states. In some embodiments, information indicating one or more psychological states of a user is received. Physiological data may be obtained from sensors, which may be attached to a user for example via a wearable device or apparatus. Allostatic load data may be obtained via biosensing lab that analyzes bio data such as saliva or blood. The system may further comprise correlating one or more data sources and information sources to provide indicate and/or manage a user's stress levels.

### FIELD OF THE DISCLOSURE

The teachings disclosed herein relate to the field of health measurement and improvement. In particular, the teachings disclosed herein relate to a system, apparatus and method for stress identification and management.

### BACKGROUND OF THE DISCLOSURE

Stress has been described by the World Health Organization as the health epidemic of the 21st century. Experts have defined stress as an internal process that occurs when a person is faced with a demand that is perceived to exceed the resources available to effectively respond to that demand, and where failure to effectively deal with the demand has important consequences.

The current methods that individuals choose for measuring and managing stress are restricted in scope, have limited versatility, and are sometimes counterproductive. Dominant techniques that attempt to manage stress include ineffective coping measures such as television watching, internet surfing, overeating, alcohol consumption and smoking.

The consequences of the inability to adequately manage stress are severe, and can be well understood through the biological mechanism of allostasis. Allostasis is the process whereby an organism maintains physiological stability by changing parameters of its internal milieu by matching them appropriately to environmental demand. Allostatic load is the ‘wear and tear’ the body experiences when repeated allostatic responses are activated during stressful situations.

Accordingly, there is a need for techniques to better improve stress monitoring and management. Therefore, there is provided a novel system, apparatus and method for individualized stress management.

### SUMMARY

In one aspect, there is provided a system for individualized stress management including a processing unit and a set of user-interfacing devices for collecting user data, the user data including biological data and physiological data; wherein the set of user interfacing devices are connected to the processing unit to transmit the collected user data for determination of individualized stress management; and wherein the biological data includes allostatic load data. In another aspect, the user data includes psychological data.

In another aspect of the disclosure, there is provided a method of stress management including performing an initial stress assessment based on a set of initial stress assessment measurements; and recommending a stress intervention program based on the initial stress assessment by comparing the set of initial stress assessment measurements with a set of baseline measurements; wherein each of the set of initial stress assessment measurements and set of baseline measurements include biological data and physiological data, the biological data including allostatic load data. In another aspect, the set of baseline measurements includes psychological data.

### BRIEF DESCRIPTION OF DRAWINGS

It is understood that persons having skill in the relevant understanding that the figures included herein are designed to convey important technical features of the teachings disclosed herein. In some instances, dimensioning and/or orientation may be presented such that principles of one or more aspects of the present disclosure may be better conveyed. Further, certain components or pieces that may be incorporated into commercial implemented embodiments may not be presented so that other features of the embodiments of the present disclosure may be less obscured.

FIG. 1 shows the pathophysiological process of allostatic load and the multi-system biological dysregulation caused by chronic stress;

FIG. 2 illustrates the interplay between environment, individual differences, perceived stress levels and behavior responses on allostatic load;

FIG. 3 is a flowchart of one embodiment for individualized stress management in accordance with an embodiment of the present disclosure;

FIG. 4a is a schematic diagram of a system for stress management in accordance with an embodiment of the present disclosure;

FIG. 4b is a schematic diagram of another embodiment of a system for stress management;

FIG. 4c is a schematic diagram of a component of the system for stress management;

FIG. 5a shows a user interacting with a biological data apparatus of the system for stress management;

FIG. 5b is a schematic diagram of another embodiment of a biological data apparatus of the system for stress management;

FIGS. 6a to 6c are perspective views of a physiological apparatus for use with the system for stress management;

FIGS. 6d to 6i are line drawings of the physiological apparatus of FIGS. 6a to 6c;

FIG. 6j is a perspective view of a hydrogel electrode for use with the physiological apparatus;

FIG. 6k is an exploded view of the hydrogel electrode of FIG. 6j;

FIG. 7 is a table outlining a list of sensors for use with the physiological apparatus;

FIG. 8 is a schematic view of a portion of a psychometric apparatus; and

FIGS. 9a to 9c are example charts outlining data collected by the system for stress management.

Skilled artisans will appreciate that elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present disclosure. Also, common but well-understood elements that are useful or necessary in a commercially feasible embodiment may not be depicted in order to facilitate a less obstructed view of these various embodiments of the present disclosure. It will further be appreciated that certain actions and/or steps may be described or depicted in a particular order of occurrence while those skilled in the art will understand that such specificity with respect to sequence is not actually required. It will also be understood that the terms and expressions used herein have the ordinary technical meaning as is accorded to such terms and expressions by persons skilled in the technical field as set forth above except where different specific meanings have otherwise been set forth herein.

### DETAILED DESCRIPTION OF THE DISCLOSURE

The disclosure is directed at a system, apparatus and method of individualized stress management. In one embodiment, the disclosure may use information or data associated with a user to assess a stress level of the user, identify one or more interventions for the user and/or adopt behaviors for the well-being of the user. In the preferred embodiment, the system uses at least one of biological data, physiological data and psychometric data to providing the individualized stress management.

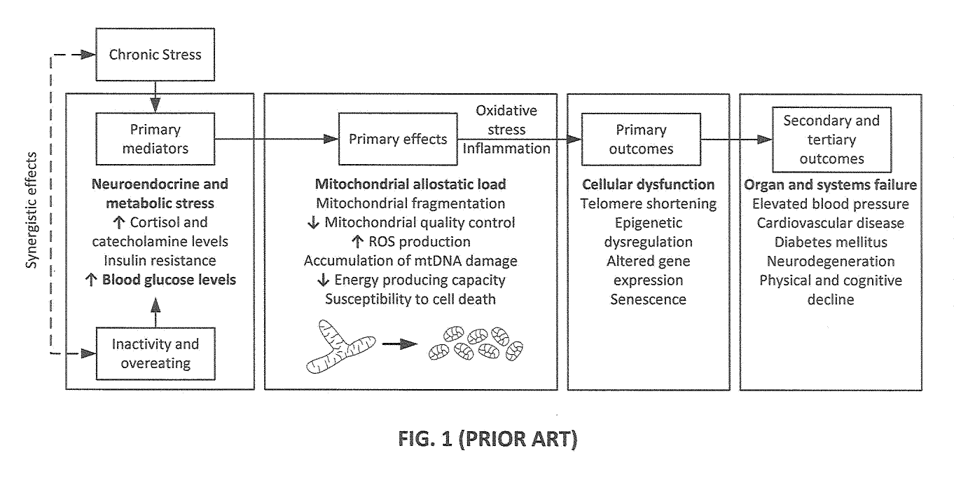

FIG. 1 illustrates a series of downstream effects and outcomes that result from chronic stress, as described in McEwen B S, McKittrick C R, Tamashiro K L K, Sakai R R The brain on stress: Insight from studies using the Visible Burrow System PHYSIOLOGY & BEHAVIOR 2015 Jul. 1; 146:47-56. The body attempts to mediate the experience of stress with various systems, including the endocrine system, which releases hormones (such as cortisol) and catecholamines (such as epinephrine and norepinephrine).

The allostatic load impacts the body on the subcellular level, with components such as mitochondrial function adversely impacted and damage inflicted upon mitochondrial DNA (mtDNA). Significant cell damage and even cell death increases with the production of reactive oxygen species (ROS) molecules. The outcome of these biological processes is cellular dysfunction and problematic genetic regulation and disregulation, which in turn manifest in more macro-components of the human body such as organ failure and system deterioration ranging from cognitive decline to diabetes.

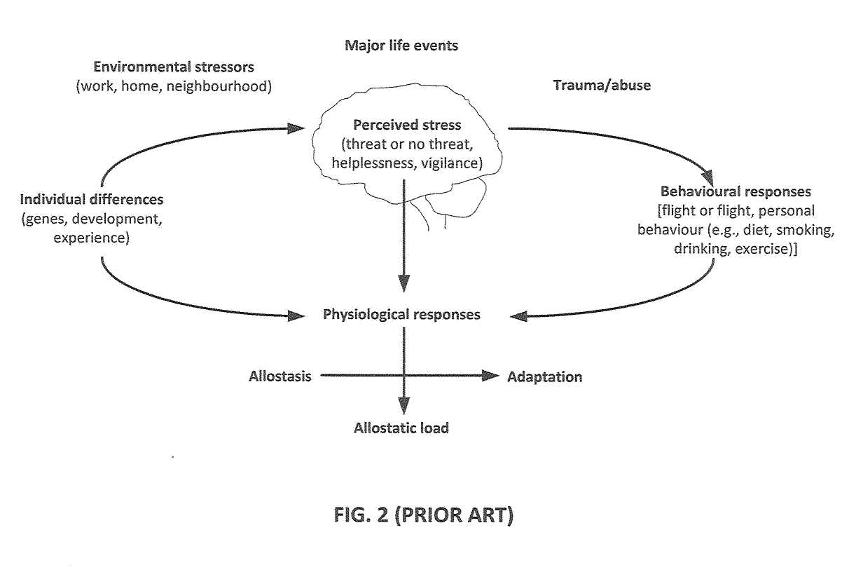

Fundamental influences on the initiation, propagation and/or mitigation of stress can be seen in FIG. 2 as discussed in McEwen B.; Allostasis and Allostatic Load: Implications for Neuropsychopharmacology; 2000; page 114, Neuropsychopharmacology-Vol. 22, No. 2. External stressors may be produced from the surrounding environment or can be physically or verbally imposed. How, and to what degree, an individual perceives those external stressors are based on both a) individual traits such as unique genetics and b) developmental experiences that influence genetic and epigenetic expression. These individual differences, in tandem with an individual's perceived stress levels and behavioral response (e.g., smoking when stressed), affect the physiological response of the individual to stressors. The physiological response, as explained above, result in the body undergoing allostasis to adapt to the stress, which in turn yields allostatic load ‘wear and tear’.

As understood, the detrimental impacts of stress on society indicates that individuals are inadequately managing stress through subjective assessments, such as self-perceived feelings of stress. Self-detection approaches alone lack precision, as it is difficult to determine whether a given perceived feeling of stress indicates normal stress levels that are unlikely to produce adverse health effects—or alternatively abnormal levels that run the risk of obesity, heart disease, and other such problems. Self-detection approaches also fail to provide timely indications of stress, as maladaptive hormone levels may only manifest as physically perceivable system after years or decades, at which time significant and irreversible damage has already occurred.

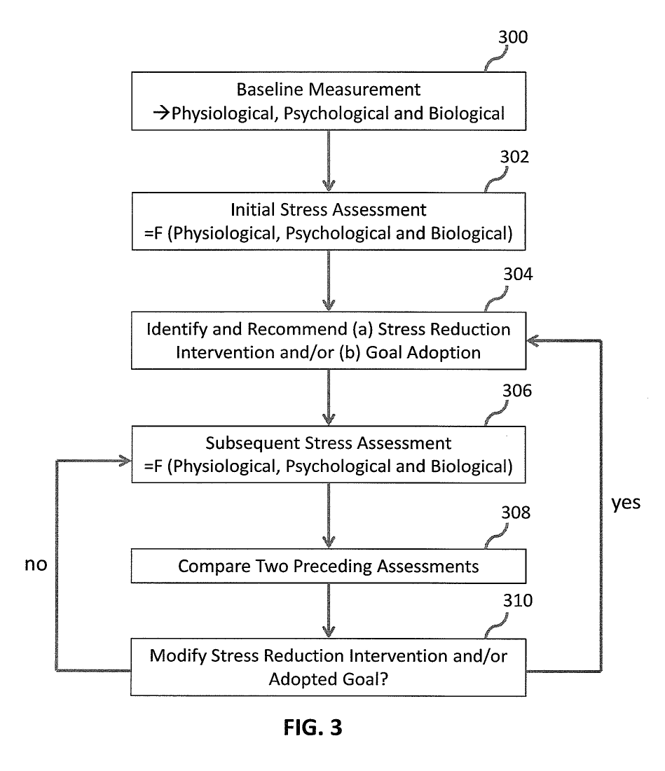

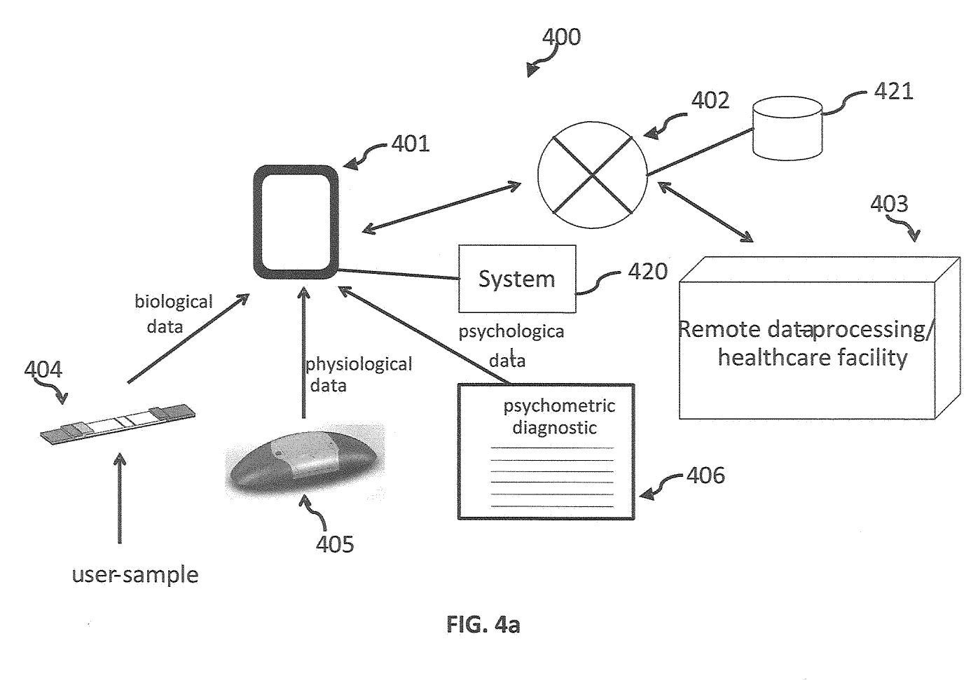

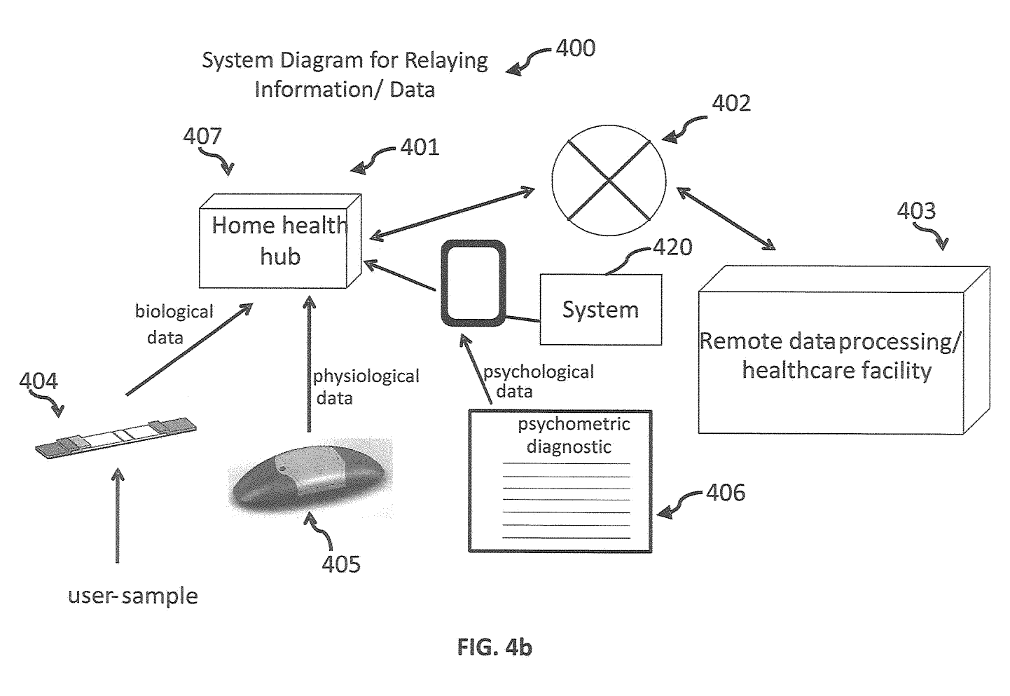

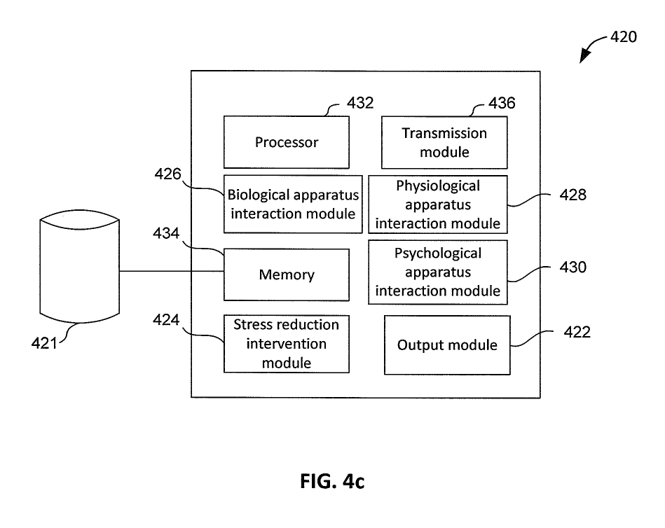

Turning to FIG. 4a, a first embodiment of a system for individualized stress management is shown. In the embodiment of FIG. 4a, the system is used to obtain dimensions or data to assess stress levels and identify intervention/goals for stress management. A method of improving a user's stress management is described below with respect to FIG. 3.

The system 400 includes a processing unit 401, seen as a client computer that connects via a network 402 to a remote data-processing or healthcare facility 403 and a database 421. The network and the healthcare facility may or may not form part of the system for individualized stress management. The system 400 further includes various components which transmit user-related data to the client computer 401. These components, which may collectively be seen as user-interfacing apparatus, include, but are not limited to, biological data apparatus, seen in the current embodiment as a mini-biolab 404, physiological data apparatus, seen in the current embodiment as a wearable patch device 405 and psychological data apparatus, such as in the form of a psychometric diagnostic 406. The psychometric diagnostic 406 may be input by the user into a computer or the like which then transmits the results to the client computer 401 or responses to the diagnostic may be input directly by the user into the client computer 401. Each of the mini-biolab 404, the wearable patch device 405 and the psychometric diagnostic 406 obtain relevant data and information from a user and transmits the data to client computer 401 for processing and the determination of individualized stress management or an individualized stress management plan. The client computer 401 may also be connected to an apparatus for determining stress management 420.

Client computer 401 may be any suitable client computing device such as a rack-mounted computing device, a desktop computing device, a laptop computing device, a tablet computing device, a phone-tablet (phablet) computing device, a mobile device such as a mobile cell-phone, a smartphone or a personal digital assistant (PDA), or wearable computing device such as a smart-watch or smart computer band worn on the user's extremity, integrated into glasses or otherwise appended to a user's body or garment. The computing device may have one or more connecting interfaces that may be used to link the computing device 401 to or allow the computing device to communicate with other devices such as a display screen, a projector, speakers, headphone or other such output devices capable of video and/or audio generation. The computer device may also have one or more connecting interfaces that allow for interconnection with various networks 402 such as, but not limited to, a local area network, wide area network, other specialized network (whether publicly open or securely closed), the internet or to other computing devices including remote data-processing/healthcare facility 403.

Turning to FIG. 4c, the apparatus for determining stress management is shown. Although shown outside the client computer, the apparatus 420 may also be integrated within the client computer 401. The system 420 includes an output module 422, a stress intervention module 424, a biological data apparatus interaction module 426, a physiological apparatus interaction module 428 and a psychological apparatus interaction module 430. The system 420 also includes a processor 432, a memory module 434, and a transmission module 436.

The output module 422 is configured to create or produce display screens which are to be displayed on the client computer 401 depending on the action being performed by the user or based on information to be delivered to the user, such as a stress management plan. The output module communicates with the processor 432 to obtain the content for the display screen or page.

The stress intervention module 424 determines the individualized stress management plan based on the information or data supplied by the biological data apparatus interaction module 426, the physiological apparatus interaction module 428 and psychological apparatus interaction module 430. The stress intervention module 424 communicates with the processor 432 or the output module 422 or both to determine the information to be delivered for display on the client computer by the system 420.

The biological data apparatus interaction module 426 is configured to receive information or data which is input from the biological data apparatus. The physiological apparatus interaction module 428 is configured to receive information or data which is input from the physiological data apparatus while the psychological apparatus interaction module 430 is configured to receive information or data which is input from the psychological data apparatus or to receive direct input from a user. The data that is collected from these modules is then transmitted to either the processor 432 or the stress intervention module 424 for assisting in determining an individualized stress management plan. Although shown as separate modules, the biological data apparatus interaction module 426, the physiological apparatus interaction module 428 and psychological apparatus interaction module 430 may be combined as a single module.

The processor 432 is configured to execute instructions from the other modules of the system 420. In some cases, the processor 432 may be a central processing unit. In other cases, each module may be operatively connected to a separate processor. The system further includes a memory module 434, for example a database, random access memory, read only memory, or the like.

The transmission module 436 is configured to receive and transmit data to and from the network 402 or the like and may be, for example, a communication module configured to communicate between another device and/or the network 420.

Turning to FIG. 4b, another embodiment of a system for individualized stress management is shown. In this embodiment, a hub, seen as home health hub 407, acts as a relay station between the biological data apparatus (mini-biolab 404), the physiological data apparatus (wearable patch device 405) and the psychological data apparatus (psychometric diagnostic 406) to the network 402. In the current embodiment, the client computer 401 interacts with the home health hub 407 to transmit or deliver the psychometric diagnostic 406. The health hub may receive and organize data from the user-interfacing apparatus and arrange the data for identification of patterns. In one embodiment, the home health hub may be programmed to identify/assign timestamps for received user data, and arrange and organize data from several user-interfacing devices according to a timeline. Such a timeline may be the basis for identifying anomalous events or periods of stressful activity. For example, a stressful event may be identified based on increased cortisol readings from mini-biolab 404, reduced heart rate variabilities (HRV) readings from wearable patch device 405, and reported feelings of anxiety or depression from a survey questionnaire in psychometric diagnostic 406. In some embodiments, such a data analysis may occur at the home health hub 407 while in other embodiments, analysis occurs either at client computer 401 or the remote data processing/healthcare facility 403.

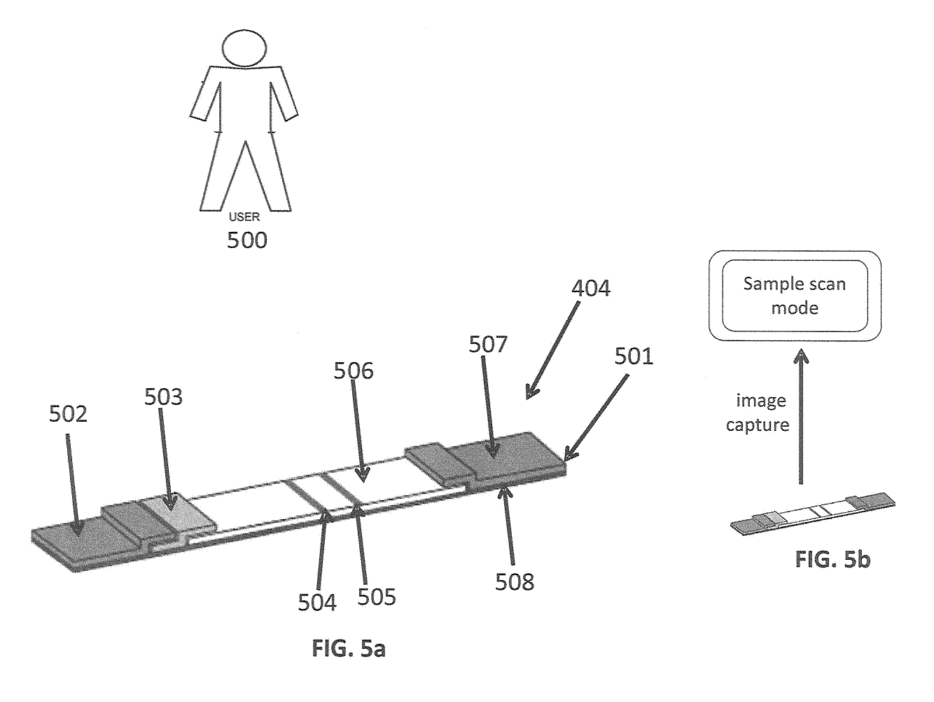

Turning to FIG. 5a, an embodiment of a biological data apparatus is shown. The biological data apparatus of FIG. 5a may be seen as the mini-biolab of FIG. 4a or 4b. The biological data apparatus is used to obtain a bodily tissue and/or fluid sample (biosample) from a user 500. In the current embodiment, the mini-biolab 404 is used for biological sensing of the biosample to provide data for an individualized stress management plan. In one embodiment, this may be performed by identifying allostatic measurements. After obtaining the biosample from the user, the biosample is placed on a sample pad 501 of the mini-biolab 401 either by the user 500 or by another party. The sample pad 501 may be seen as a bio-strip or a bio-chip.

The sample pad 501 includes a conjugate pad/detection conjugate 503 along with a membrane 506 where an amount of texture and visual appearance change of membrane 506 is designed to vary based on the target analyte (biomarker) that is being tested. Atop the membrane are a test line 504 and a control line 505. An absorbent pad 507 is also present to capture any residual of the biosample. In some embodiments, the sample pad 501 may also include a plastic adhesive portion 508.

To obtain the biosample, a lancet or other known methods may be used to prick a user's finger or other body part to obtain a blood sample. In addition, or alternatively, to the blood sample, a saliva sample may be obtained. Additionally, a vial may be used to obtain the biosample with the user spitting into the vial to capture the saliva. In some embodiments, the user may spit on or lick the sample pad 501.

Sweat is another biosample that may be obtained from the user and placed on the sample pad 501. In one embodiment, the sweat biosample may be obtained by swiping off sweat with a cotton swab or q-tip and then transferred to the sample pad 501. Additionally or alternatively, areas of skin moist with sweat, or beads of sweat perspiring from the skin, may be obtained by placing the sample pad 501 directly in contact with the sweat or the user's skin. In yet another embodiment, the biosample may be obtained via a user's tears. Tears may be produced through saline drops, which may stimulate tear production for gathering of the tear by a cotton swab, q-tip, or directly onto the sample pad 501.

Output from the excretory system may also be used as for the biosample with the output being placed on the sample pad 501 for analysis. In addition, bio-samples may be obtained from internal portions of the body in manner similar to those for obtaining samples on which biopsies are performed.

Internal bodily samples may be obtained from inner layers of the skin, various organs, bone structures, cartilage or connective tissue regions and other anatomical locations from which useful bio-data may be gathered.

While the foregoing embodiments for obtaining biosample describe a user obtaining his or her own biosample, it should be noted that the methods disclosed herein may be carried out with another party obtaining the biosample and then placing the biosample on the sample pad 501.

After obtaining the biosample, and placing it on the conjugate pad/detection conjugate 503 of the sample pad 501, analysis of the biosample may be conducted. In one embodiment, the sample pad may be an apparatus for performing a lateral flow immunoassay that acts as a platform to perform a number of tests. The chemical composition of the biosample is picked up by the conjugate pad/detection conjugate 503 which then permeates through membrane 506, where the amount of texture and visual appearance change of the membrane 506 is designed to vary based on the target analyte (biomarker) that is being tested. For instance, if cortisol is being tested, a more significant amount of cortisol in the biosample will cause membrane 506 to have a larger amount of its surface area change in color than a sample with a smaller amount of cortisol. The degree to which the membrane changes color past test line 504 and how close to control line 505 the surface color of the membrane changes indicates the level of cortisol present in the biosample Any residual sample may then be absorbed by absorbent pad 507.

In some embodiments, examination of the lateral immunoassay strip may be conducted through manual visual inspection by looking to see how far along the length of membrane 506 has changed color. Such inspection may occur with the aid of reference strips, where the reference strip that most resemblances the membrane corresponds to a particular biomarker measurement level. This data may then be transmitted to the computing device 401 of FIG. 4a or the home health hub 407 of FIG. 4b. The data may represent a length of colour change or a percentage of the membrane that has changed colour.

In another embodiment, automated image processing methods are contemplated. As illustrated in FIG. 5b, a digital image capture of the membrane 506 of the biological data apparatus may be obtained and the pixels within the digital image capture analyzed to determine where the membrane color alteration has taken place relative to test line 504 and control line 505. This analysis may be performed by a computing device associated with the sample pad 501 or the digital image may be transmitted to the client computer 401 or the home health hub 407 for such analysis and processing. The digital image may be captured through any suitable photographic or video-capture apparatus available on any device including cameras or optical recognition devices. The distance that the biosample has caused the membrane to change texture or color corresponds with particular biomarker levels. For example, the greater the proportion of membrane length that has been changed color by the biosample may represent that the biosample contains or contained a higher level of cortisol.

Other embodiments of mini-biolab 404 may include placing the biosample on a sample pad which is then inserted into a bio-lab device designed to receive and potentially supply power to the sample pad such as a bio-chip. The bio-chip can then transmit enzyme data where further analysis may be performed as described below. In embodiments that employ a biochip with a received housing or platform, enzyme levels may be determined using electrochemical bio-sensing using micro-fluidic processes. Additionally or alternatively, chemoimmunillescence through a photographic image of the sample may be performed. Each of these may be seen as a part of or a stand-alone biological data apparatus.

Furthermore, analysis of the biosample may be achieved via single-plexing or multiplexing. In the instance of single-plexing, levels of a single analyte may be assessed or tested while, by contrast, two or more analytes may be tested for when multiplexing is performed. The analysis being performed may produce biomarker data such as, but not limited to, neuroendocrine data, immune metabolic data, and cardiovascular and respiratory function data. The following table represents a listing of various biomarker data (organized by biomarker type) that may be measured in the analysis performed by mini-biolab 404.

| Type | Biomarker | Function |
|---|---|---|
| NEURO- | Cortisol | Glucocorticoid produced by |
| ENDOCRINE | | the adrenal glands. |
| | | Functions include the |
| | | conversion of stored fats and |
| | | proteins into carbohydrates, |
| | | anti-inflammatory and |
| | | immuno-suppressive effects, |
| | | increased blood pressure and |
| | | heart rate, suppression of |
| | | digestive, growth, and |
| | | reproductive activities, and |
| | | modulation of limbic and |
| | | prefrontal regions upon |
| | | traversing the blood-brain |
| | | barrier. |
| NEURO- | Dehydro- | Androgen produced by the |
| ENDOCRINE | epiandrosterone | adrenal glands. Known |
| | | functions include its role as a |
| | | HPA-axis antagonist and its |
| | | ability to convert into |
| | | androgens and estrogens. It |
| | | also suppresses inflammatory |
| | | cytokines, improves lipid |
| | | metabolism and lean muscle |
| | | mass, decreases insulin |
| | | resistance, and reduces |
| | | oxidative brain damage. |
| NEURO- | Epinepherine | Catecholamine produced by |
| ENDOCRINE | | the adrenal glands and the |
| | | brain. As part of the “fight-or- |
| | | flight” response, it increases |
| | | heart rate and glucose levels |
| | | while decreases digestive |
| | | and immune functions. |
| NEURO- | Norepinepherine | Catecholamine produced by |
| ENDOCRINE | | the brain. As part of the |
| | | “fight-or-flight” response, it |
| | | increases blood pressure, |
| | | constricts blood vessels, and |
| | | modulates brain activities. |
| NEURO- | Dopamine | Catecholamine produced |
| ENDOCRINE | | primarily in the brain and |
| | | adrenal glands. It is a well- |
| | | characterized |
| | | neurotransmitter involved in |
| | | many neurological activities |
| | | (motivation, voluntary |
| | | movement, cognition) and |
| | | also increases blood |
| | | pressure and heart rate. |
| NEURO- | Aldosterone | Minerocorticoid produced by |
| ENDOCRINE | | the adrenal glands. |
| | | Functions by reabsorbing |
| | | sodium, retaining water, and |
| | | excreting potassium in the |
| | | kidneys in order to maintain |
| | | blood acidity, as well as to |
| | | decrease blood volume and |
| | | blood pressure. |
| IMMUNE | Interleukin-6 | Cytokine produced by |
| | | macrophages and T-cells. |
| | | Functions in pro-inflammation |
| | | and anti-inflammation by |
| | | stimulating B cell and T cell |
| | | differentiation that assist |
| | | acute phase reactions to |
| | | tissue damage. |
| IMMUNE | Tumor necrosis | Cytokine produced by |
| | factor-alpha | macrophages. Functions in |
| | | systemic inflammation by |
| | | evoking mediators of acute |
| | | phase reactions as well as in |
| | | tumor apoptosis. |
| IMMUNE | C-reactive protein | Protein synthesized in the |
| | Insulin-like | liver. Functions by enhancing |
| | growth factor-1 | phagocytosis during acute |
| | | phase reactions that promote |
| | | inflammation. |
| IMMUNE | Insulin-like | Polypeptide protein hormone |
| | growth factor-1 | produced primarily in the liver |
| | | and pancreas. Functions as |
| | | a stimulator of cell growth |
| | | and as an inhibitor of cellular |
| | | apoptosis. |
| IMMUNE | Fibrinogen | Protein that synthesizes into |
| | | fibrin in the liver. Upon |
| | | synthesis, functions as a |
| | | blood clotting factor that |
| | | promotes coagulation but |
| | | when excessive increases |
| | | risk of thrombosis. |
| METABOLIC | High density | Lipoprotein synthesized in the |
| | lipoprotein | liver. Transports cholesterol |
| | cholesterol | from tissues to the liver. |
| | | Commonly referred to as |
| | | “good cholesterol”, as its |
| | | high protein/low cholesterol |
| | | form is more easily removed |
| | | by blood in the liver and |
| | | excreted in bile. |
| METABOLIC | Low density | Lipoprotein synthesized in the |
| | lipoprotein | liver. Transports cholesterol |
| | cholesterol | to tissues that synthesize cell |
| | | membranes and secretions. |
| | | Commonly referred to as |
| | | “bad cholesterol”, as its low |
| | | protein/high cholesterol form |
| | | is more likely to be deposited |
| | | in the walls of blood vessels |
| | | and contribute to |
| | | atherosclerosis. |
| METABOLIC | Triglycerides | Glyceride formed from |
| | | glycerol and three chains of |
| | | fatty acids. Functions as an |
| | | important source of energy |
| | | and as a transporter of |
| | | dietary fat. |
| METABOLIC | Glycosylated | Hemoglobin used to index the |
| | hemoglobin | average glucose |
| | | concentration over many |
| | | days, weeks and even |
| | | months. This proportion |
| | | represents the amount of |
| | | glucose that the analyzed |
| | | hemoglobin has been |
| | | exposed to during its cell |
| | | cycle. |
| METABOLIC | Glucose | Monosaccharide synthesized |
| | | in the liver and kidneys. |
| | | Functions as our main source |
| | | of energy. |
| METABOLIC | Insulin | Protein hormone produced in |
| | | the pancreas. Functions by |
| | | lowering glucose levels and |
| | | promoting energy storage in |
| | | the form of glycogen. |
| METABOLIC | Albumin | Protein produced by the liver. |
| | | Functions in the maintenance |
| | | of blood volume regulation |
| | | and as carrier for molecules |
| | | of low water solubility. |
| METABOLIC | Creatinine | Nitrogeneous waste product |
| | | of muscle creatine phosphate |
| | | that is filtered and excreted |
| | | by the liver. Creatinine |
| | | clearance is a marker of |
| | | glomerular filtration rate |
| | | representing renal |
| | | functioning. |
| METABOLIC | Homocysteine | Amino acid biosynthesized |
| | | from methionine and can |
| | | convert into cysteine. |
| | | Functions in remethylation |
| | | and transsulfuration |
| | | pathways that are in part |
| | | dependent on nutritional |
| | | intake of folic acid and |
| | | vitamin B12. |
| | | Excessive homocysteine |
| | | levels have been implicated |
| | | in risk of cardiovascular |
| | | disease. |

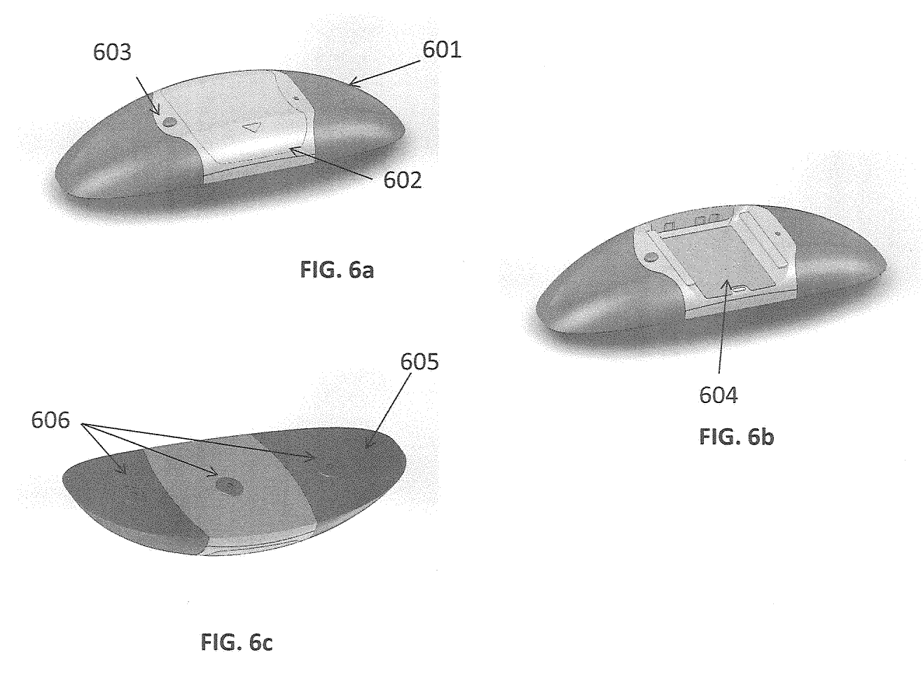

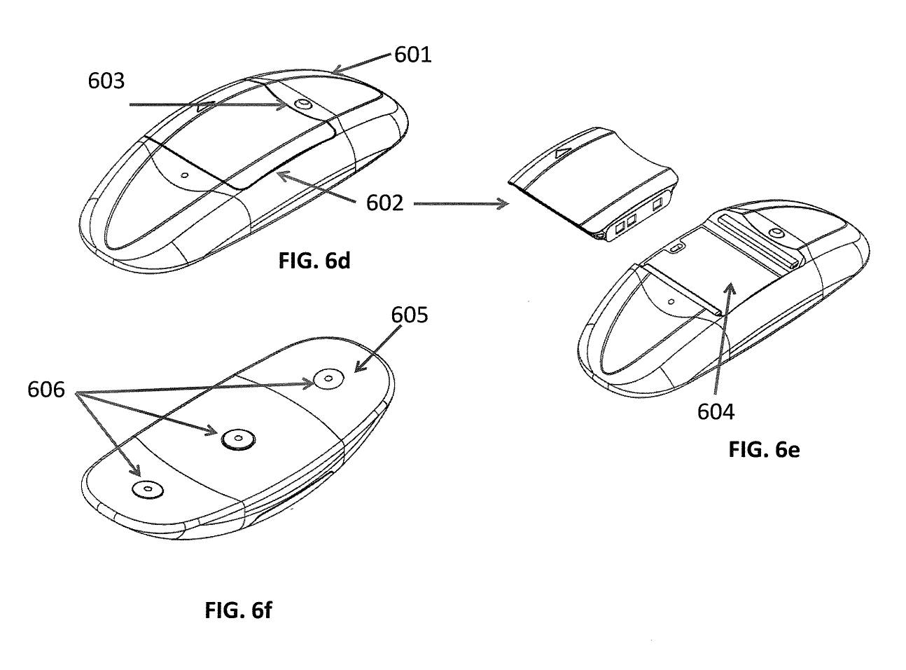

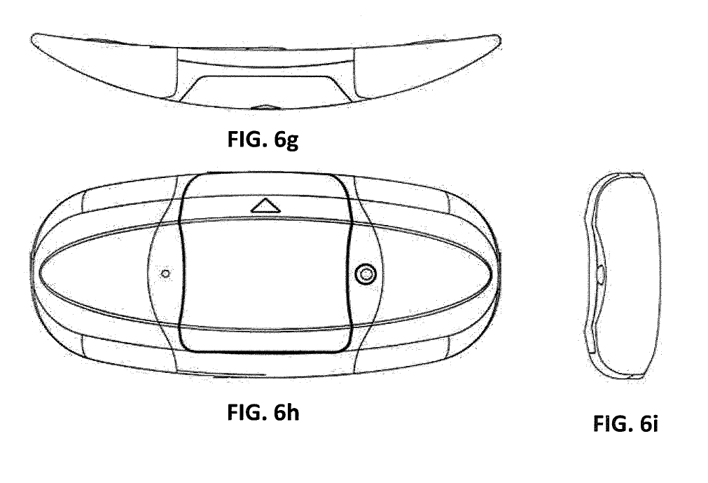

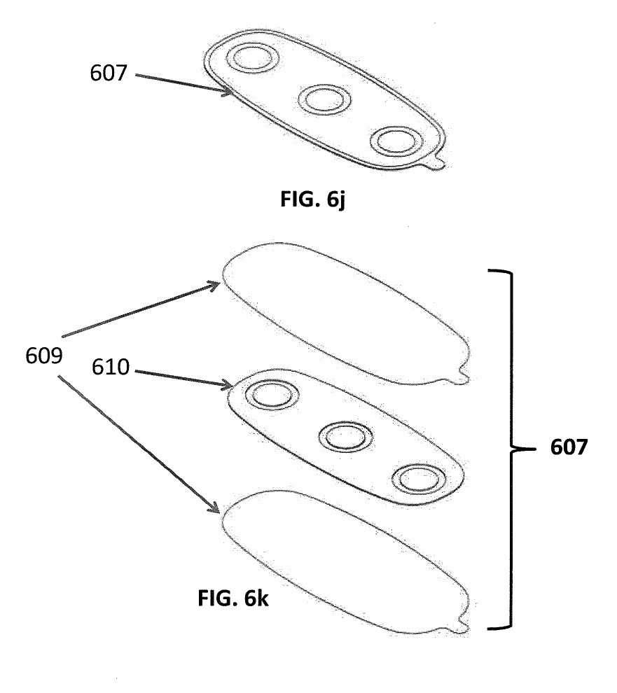

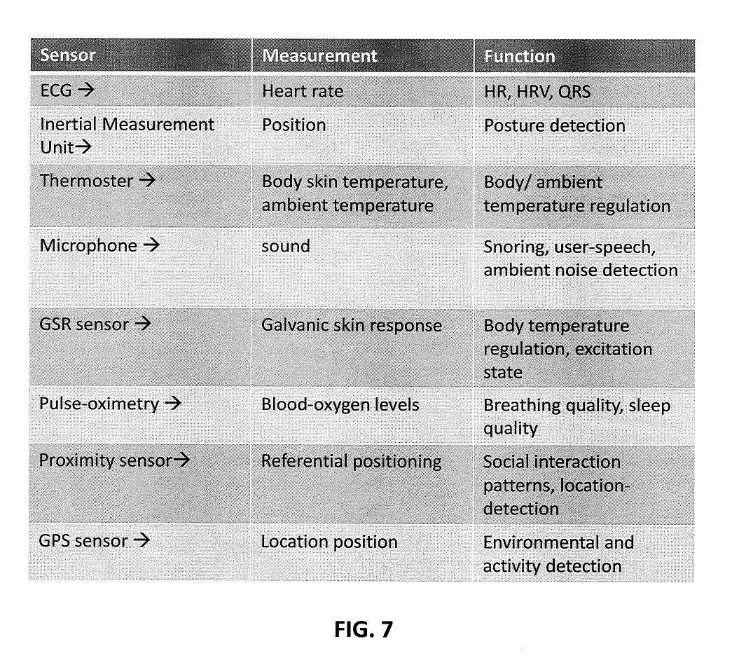

Turning FIGS. 6a to 6c, various views of a physiological data apparatus are shown. In the current embodiment, the physiological data apparatus is a wearable patch. The wearable patch functions as physiological sensor and is configured to measure physiological data associated with a user with various sensor units, as further described below. In FIG. 6a, a perspective top view of a surface of the wearable patch that is exposed when the wearable patch is adorned to the body of a user is shown. The wearable patch device 405 includes a shell 601 which is constructed from any suitable elastic material that enables the patch 405 to flex upon being fixed to the body surface of a user. The patch 405 includes a battery 602, such as a modular battery. In use, the modular battery may be removed from the shell 601 of the patch. In some embodiments, depressing a release button 603 may assist in releasing a secured modular battery 602. The modular battery 602 may be secured into a housing area 604 (FIG. 7b) within the patch through any number of mechanisms known to those of ordinary skill in the art such as, but not limited to, the use of snap-fitting, latching mechanisms, thread-and-screw mechanisms.

Turning to the perspective view of FIG. 6b, the wearable patch device 405 is shown with the modular battery removed. The shell 601 includes the housing area 604 which receives the battery. FIG. 6c is a bottom perspective view which shows a body contact side or surface 605 of the wearable patch. As can be seen in these figures, the surface on which modular battery is housed is preferably different than the surface which is in contact with the user's body such that the modular battery may be switched or replaced without having to remove the wearable patch from the user's body.

The body contact side 605 preferably includes an elastic surface or is made from a material that flexes to conform to the user's body when the user's body expands or contracts. The body contact side 605 may include a conductive layer that enables transmission of electrical signals such as biometric signals emitted from the user. The body contact surface 605 also includes one or more electrodes (or sensors) 606 that are capable of detecting transmitted biometric signals, as described below.

FIGS. 6d to 6i are further drawings showing various views of the wearable patch of FIGS. 6a to 6c.

Turning to FIG. 6j, a perspective view of a disposable electrode sheet for use with the wearable patch is shown while FIG. 6k provides an exploded view of the disposable electrode sheet. A portion of the disposable electrode sheet 607 is placed on the body contact surface 605 of the wearable patch prior to having the wearable patch being placed into contact with the user's body. The electrode patch 607 preferably includes a pair of release, or protective, liners 609 which sandwich an adhesive liner, or layer, 610 such as, but not limited to an adhesive hydrogel layer. The release liners 609 protect the adhesive properties of the adhesive hydrogel layer 610 prior to the adhesive layer 610 being adhered to the body contact side 605 of the wearable patch device 405. Although only a single hydrogel liner is shown, the hydrogel electrode may include any number of adhesive layers. Once separated from release liners 609, the adhesive hydrogel layer 610 is affixed to the body contact surface 605 of the wearable patch device 405. Both sides of the hydrogel layer preferably include an adhesive such that the wearable patch device 405 can then be placed in contact with and adhered to the user's body. These adhesive layers may be replaced, as needed. The adhesive layer 610 conducts biometric signals to the electrode 606 housed within the wearable patch device 405. In one embodiment, since the hydrogel electrode has conductive properties, the electrical signals emitted by the user are sensed and then transmitted to the system. The layer includes holes which align with the sensors of the wearable patch 405.

Turning to FIG. 7, a table showing different types of sensors which may be included in wearable patch device 405 is shown. In one embodiment, the sensors 606 may be electro-cardiogram (ECG) sensors to measure heart beats in order to calculate the user's heart rate, heart rate variability and/or QRS complex assessments. In another embodiment, one of the sensors 606 may be an inertial measurement unit (IMU) to detect user movements and positions, and determine postures or the user to assist in determining if improvements may be made by the user. In yet a further embodiment, the sensors 606 may be a thermistor that measures body skin temperature and ambient temperature in order to assist in identifying fluctuations in either the skin temperature or the ambient temperature and the impacts thereof on the health of the user. Another example of a sensor 606 may be a microphone to obtain sound measurements to identify snoring, user-speech, individuals with whom the user is conversing with and detecting ambient sound detection either natural (e.g., thunder) or man-made (e.g., traffic). Another type of sensor 606 that can be used is a pulse-oximetry sensor that can measure blood-oxygen levels to assist in determining breathing quality or sleep quality (that may be impacted by improper breathing with apnea-related problems). Furthermore, the sensor 606 may be a proximity sensor to measure referential positioning so that social interactions with others or proximities to objects that may impact user-physiology, biology or psychology can be assessed. Other sensors may be integrated within the wearable patch such as, but not limited to, a global positioning (GPS) sensor to provide location information to identify the user movement patterns, environmental/activity as further described below. Other integrable sensors include an accelerometer or a proximity sensor. In a further embodiment, more than one type of sensor may be included in the wearable patch device 405.

Along with the wearable patch device 405, other physiological apparatus to obtain physiological data are contemplated whether they are integrated with the wearable patch device 405 or a separate component. These apparatus include, but are not limited to, an electroencephalogram (EEG) sensor to measure brain wave activity, an electrooculography (EOG) sensors (to measure eye activity), hydration sensor (to determine if a user is hydrated or dehydrated), glucose sensors (to detect blood sugar levels), a breath analyzer monitor (to detect blood particulate constituents), a blood pressure monitor, plethysmographs (to measure volumetric changes in the lungs, limbs, etc.), respiration sensors (to measure breathing via abdominal/thoracic expansion), electro-dermal sensors (to measure skin electrical activity), fluid detector or analyzers (to assess interstitial fluid, blood, cerebrospinal fluid, saliva, or other biological fluids) and implanted or implantable sensors. Additionally, environmental or circumstantial data gathering apparatus which gather data that may affect a user but are not directly related to a user are contemplated as physiological data apparatus. This includes, but are not limited to, a barometer (to measure the effect of atmospheric pressure that may correlate with reported sinus changes for example), weather reports for current locations (to identify how sun, temp, wind, precipitation, etc. affect physiology), barometric pressure (to determine impacts on blood pressure), or climate front activity (to determine how pressure changes may affect blood viscosity for example with impacting blood sugar levels).

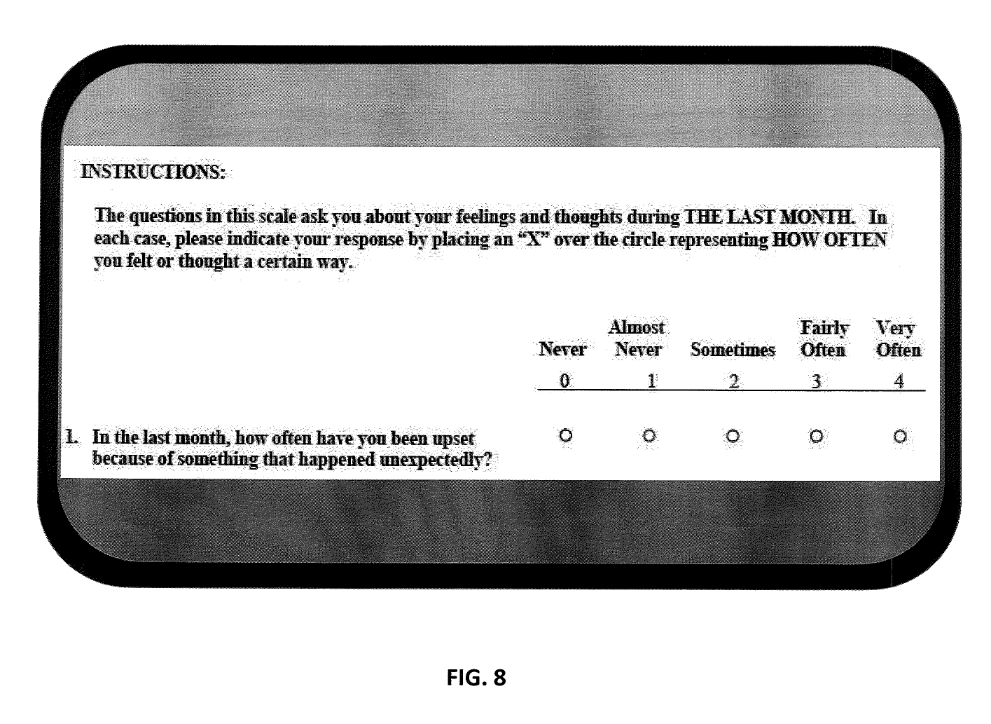

Turning to FIG. 8, a schematic diagram of a portion of a psychometric diagnosis is shown. Other example of questions are shown below in the following chart which may be seen as an exemplary psychometric assessment with Perceived Stress Scale (PSS).

```
  1. In the last month, how often have you been upset because of
something that happened unexpectedly?
  2. In the last month, how often have you felt that you were unable to
control the important things in your life?
  3. In the last month, how often have you felt nervous and “stressed”?
  4. In the last month, how often have you felt confident about your
ability to handle your personal problems?
  5. In the last month, how often have you felt that things were going
your way?
  6. In the last month, how often have you found that you could not
cope with all the things that you had to do?
  7. In the last month, how often have you been able to control
irritations in your life?
  8. In the last month, how often have you felt that you were on top of
things?
  9. In the last month, how often have you been angered because of
things that were outside your control?
10. In the last month, how often have you felt difficulties were piling
up so high that you could not overcome them?
```

The psychometric diagnostic 406 may be used to identify psychological state information that in turn may be formulated as psychological data as described above. The psychometric diagnostic information may measure the psychological coping mechanism and responses employed by a user, as well as perceived stress through a perceived stress scale (PSS). Psychological information may allow the system to assess stress from occupational activities such as workload, pyscho-social stress, social anxiety disorders, personality factors (e.g., Meyer-Briggs, DISC, etc.) intersecting with job fit (e.g., introverts performing sales functions). User responses to surveys are either presented on a computing device or handwritten responses that in turn are entered into a database are quantified and transmitted for further analysis as described below.

A number of diagnostic tools to assess psychological stress drivers in a variety of environments may be used. For example, an Occupational Stress Scale may be used to assess workplace stressors such as workload, position demands, policy strictness, time pressures, interpersonal/priority conflicts or uncertain, etc. Other such diagnostic tools may include measures of coping or resilience to assess one's style of coping and resiliency quotient. Such tools may consider the relationship(s) between various psychological dimensions to the other physiological and biological data points, as further described below.

Turning to FIG. 3, a flowchart outlining a method of individualized stress management is shown. The method is directed at assisting users in better managing the interrelated elements that initially cause, perpetuate, and ultimately exacerbate levels of stress.

Initially, a set of the baseline measurements for the user are taken (300). These baseline measurements may be seen as indications of stress levels or may be used to assess stress levels. Such measurements may include any suitable measurement relevant to stress, including, but not limited to, measurements taken via the physiological, psychological and/or biological data apparatus discussed above. The collection of these baseline measurements may not form part of the method of individualized stress management as these baseline measurements may also be obtained using previously collected data or based on known research of healthy stress levels. By way of example, a baseline stress measurement may include measuring the user's sleep every night for a predetermined period of time, such as 14 days. This baseline measurement may be presented as a table with each row representing an individual night and the number of hours of sleep achieved by the user on that given night, or alternatively may be presented as an average number of hours of sleep the user is achieving per night over the baseline measurement period (or some subset). The averages may be further segmented based on the time of week, such that sleep per night or average sleep per night on weekdays is separated from average sleep per night on weekends. The system may also identify the type of sleep (light sleep (Stages 1-2), deep sleep (Stages 3-4) and REM based on user movement as measured by the ECG, inertial measurement unit and pulse oximeter of wearable patch (405).

After the collection of the baseline measurements, an initial stress assessment is carried out (302). The initial stress assessment may be carried out by considering one or more of the baseline measurements. In one embodiment, the assessment may identify stress levels using only one or some portion of one baseline measurement of a single measurement type (biological, physiological or psychological). In other embodiments, more than one baseline measurement may be considered and/or more than one measurement type may be considered. The current disclosure contemplates where more than one measurement type may be considered may include one or more formulas used to calculate the partial/full values of the baseline measurement types. By way of example, measurements directly ascertained from the preceding baseline measurement step may be used as a stress measurement, assigning a score to the user's measured average sleep whereby the larger the deviation from a predetermined ideal sleep average (e.g., 8 hrs) is determined. Hence, the user's initial stress assessment may be deemed higher if a user has been measured to receive an average of 6 hrs of sleep per night than compared to if a user is measured to receive an average of 7.5 hrs of sleep per night, as 6 hrs of sleep per night deviates more from 8 hrs of sleep than does 7.5. Other examples of considering only one baseline measurement by considering only one of allostatic load, heart rate variability or PSS are further described below. Examples, where more than one type of measurement (e.g., biological, physiological, psychological measurements) are considered by a formula are also further described below.

The initial stress assessment (302) may also determine stress levels of the user as they relate to the remainder of the population, such as the level of stress an individual may have as a percentile of the general population, a specific age group, gender, geographic scope or socio-economic class (such as income levels or cost-of-living).

After the initial assessment of stress is made, a stress reduction intervention or goal adoption determination is performed (304) whereby a stress reduction intervention and/or goal is identified and recommended to the user. The stress reduction intervention may be made based on the initial stress assessment performed in (302), and identify an effective means of improving stress by isolating one or more aspects of behavior that can be modified to improve the user's stress level. For example, if the user is identified as regularly experiencing a spike in heart rate (as measured with the physiological apparatus) above 100 bpm at the same time of day, the system may identify that some activity or environment may be producing stress for the user at that time of day. The system may consider concurrent measurements of movement from an accelerometer or inertial measurement unit (or location from a GPS sensor) (via the physiological apparatus) to rule out the possibility that the spiked heart rate is due to the user is engaging in exercise.

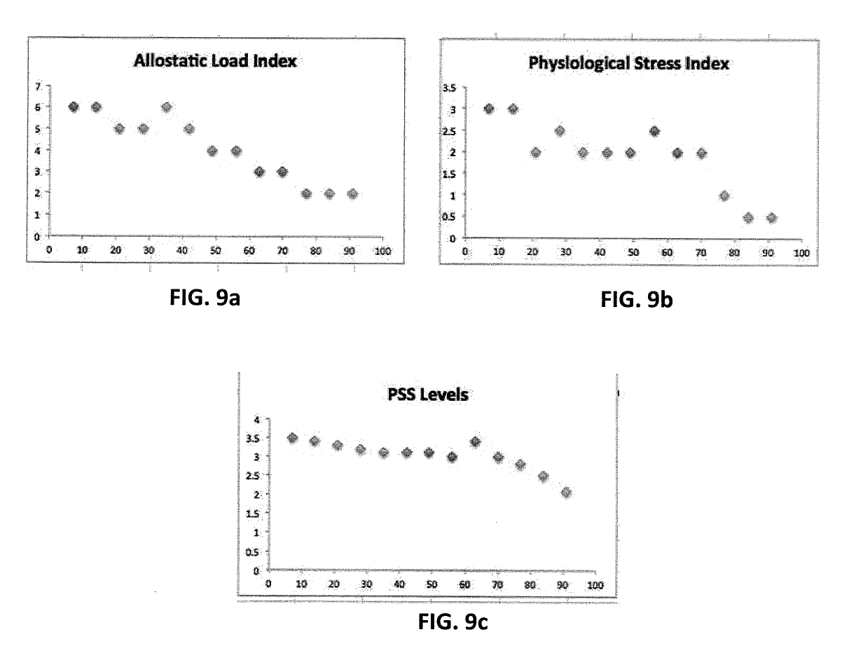

Based on identifying the recurring stressful event, such as, but not limited to, business meetings with individuals that are adversarial or a driving commute rifled with traffic, the system may recommend an intervention to reduce stress caused by that event. The intervention may be simply notifying the user that they are about to enter a stressful situation and that they should be (a) mindful of their emotional state and/or (b) engage in relaxation breathing exercises such as diaphragmatic breathing to activate the user's parasympathetic nervous system. In some embodiments, the system may have a number of pre-identified coping mechanisms that coordinate to be matched and presented upon identifying a stressful environmental cause. The treatment or suggestions may be provided to the user via a display connected to the client computer. For example, if rush-hour traffic is deemed to induce stress with a user's heart rate above a predetermined value of 100 bpm, for example, the system may identify alternative commute times or routes with reduced traffic. As further described below, driving in rush-hour traffic may be determined to be correlated with higher stress markers from psychological assessments (ratings above, for example, 20 on the PSS scale), biological assessments (group allostatic index values above, for example, 3) or a function combining several types of assessments as described below with respect to FIG. 9. The system may determine that such a correlation should result in an intervention avoiding rush-hour traffic. If interacting with one or more individuals is identified as resulting in stress, the system may suggest avoiding those individuals or provide social or interpersonal relationships techniques to improve the experience of the interaction. The intervention may also be identified by one or more individuals trained in the application of such interventions, such as certified coach, medical practitioner or other trained professional.

In some embodiments, as an alternative or in addition to a stress reduction intervention, one or more goal adoptions may be identified and recommended by the system to the user based on the initial stress assessment or the user's baseline measurements. As an example, if it is determined that a user is sleeping less than 8 hours per night on average (or the deviation in number of hours of received sleep from 8 hours is greater than a predetermined amount) and that receiving more sleep may reduce the user's stress levels, a goal of receiving at least 8 hours per night on average (or reducing the deviation in the number of hours of received sleep from 8 hours to less than a predetermine amount) may be identified and recommended to the user. Another example of a goal that may be recommended for the user to adopt based on the initial stress assessment may relate to an increased or decreased level of exercise. If the user is deemed to not exercise or exercise less than a predetermined number of times per week, the system may recommend the user adopt a goal of exercising more.

Each goal may start out at a level that is determined to be achievable but also present an appropriate level of challenge to the user. This goal may advance as the user improves his or her behavior, such that the user will be presented with successively-more challenging goals. Upon accomplishing each goal, the user may be presented with a more a difficult goal. Continuing the example above, a user whose initial stress assessment reveals that the user exercises less than once per week on average may be initially recommended to adopt a first goal of exercising twice per week. When the user has achieved consistency in exercising on average twice per week for a predetermined number of weeks, the user may then be presented with a recommended goal to adopt of exercising at least three or more times per week.

The current disclosure contemplates identifying various stress reduction interventions or goal adoptions, which may be presented as modules. A module may be defined as a unit for which a precise behavioral change is targeted. Each module may pertain to habit formation related to specific types of behaviors. For example, one module may focus on nutrition and eating habits, while another module may focus on sleep and rest habits. Modules may pertain to any number of habits, and in some embodiments each module encompasses more than one habit. Modules may deal with behavior relating to, but not limited to, cardio respiratory fitness, nutrition, sleep, breathing, relaxation, meditation, social behavior, self-compassion, financial practices, gratitude training, etc. A module pertaining to sleep, for example, may target increased average sleep periods as discussed above. A user may be presented with various modules to choose which type of behavior or specific habits the user will first focus on improving.

After a stress reduction and/or goal adoption has been identified and recommended to the user and the user has modified or attempted to modify his/her behavior, a subsequent stress assessment (306) can be conducted. The stress assessment may be similar to the stress assessment previously performed (302) but with measurements taken at a different point in time. The subsequent stress assessment may be carried out by considering one or more previously gathered measurements or by gathering new data from the physiological, psychological and/or biological data apparatus preferably taken since the stress reduction intervention and/or adoption of goals took place (304). However such measurements need not be limited to physiological, psychological and/or biological data and may include other types of measurements which assist in determining a user's stress level.

Upon completion of subsequent stress assessment (306), a comparison is made between the two preceding stress assessments (308). When the method is first executed and the comparison is performed for the first time, the two preceding stress assessments will be the subsequent stress assessment (306) and the initial stress assessment (302). Hence, the system will determine the impact on the user of having undergone the stress reduction intervention and/or goal adoption (304) by comparing (a) initial stress assessment (302) based on measurements before the stress reduction intervention and/or goal adoption with (b) subsequent stress assessment (306) based on measurements taken after the stress reduction intervention and/or goal adoption. Comparisons based on a particular behavior type, such as sleep may be compared. By way of a simple example, a user's average sleep may be compared before and after the user adopted a sleep improvement module. As another non-limiting example, cortisol or other neuroendocrine levels may be used to determine allostatic load levels before and after the stress reduction intervention (304). As discussed below, a combination of these biological and physiological factors may be considered in tandem when comparing assessments.

On the second instance of the method being executed, when the comparison (308) is being performed, there would be at least three preceding assessments: (1) the initial assessment (302), (2) the first subsequent assessment (306) from the first time the method was executed, and (3) a second subsequent assessment (306) from the second time the method was executed. In some embodiments, the comparison may include comparing the two immediately preceding assessments (e.g., the first and second subsequent assessment of 304) to determine the most recent changes. Alternatively, the comparison 308 may consider comparing the more recent assessment 306 with the initial stress assessment 302 to see what improvements have been made since the stress management system was first introduced.

The system may then modify the stress reduction intervention and/or goal that was previously adopted (310). If progress has been made but a goal or intervention has not yet been fully achieved, the system may suggest maintaining the goal or intervention and proceed perform a subsequent stress assessment (306) after further measurements are considered. In some embodiments, the degree to which progress has been made on a specific intervention or goal will be considered. By way of example, if a user has averaged gym visits more on average per week since a goal adoption, but still needs to visit the gym with greater frequency to meet the goal, the system may suggest maintaining and not modifying the goal. If minimal or no progress has been made, a series of diagnostic questions may be introduced to identify why the desired behavior change has not been accomplished (e.g. is the user still motivated to improve that behavior, was an goal too difficult, were there outside circumstances that impeded the effects of an intervention, etc.) Based on the results of the diagnostic inquiries, the system may suggest the user continue to pursue the goal or adhere to the intervention, or alternatively suggest moving onto to another module. In some embodiments, the user will make the decision between moving on to a different module and proceeding or maintaining a current module.

If a goal with respect to a particular module has been met, the system may recommend modification to the stress reduction intervention and/or adopted goal and suggesting the user adopt the next most challenging goal that is of the same behavior type or category of the goal that has just been achieved. By way of example, if the user has met an adopted goal of achieving two gym sessions on average per week, the user may be recommended to adopt a goal for achieving three gym sessions on average per week. Alternatively or in addition, a module may be presented that is of a different behavior type or category than the stress intervention and/or goal adopted previously adopted by the user. For instance, if the user's previous goal was to regularly go to sleep and wake up at the same early time of day, a modified stress reduction intervention may be to introduce a goal of meditation to the newly available time in the morning. Another example may include moving on from a module pertaining to diet improvement to a module pertaining to exercise.

In one embodiment, in order to perform the stress assessments (302 or 306), the stress assessments may be conducted for biological data (such as allostatic load), physiological stress data and psychological stress data. With respect to allostatic load data, cortisol or any neuroendocrine levels that may be obtained with biological sensor measurements from the biological DATA apparatus 404 to make an allostatic load determination. Individual biomarker levels may be used or alternatively several biomarkers may be combined to form an allostatic load determination. Further, allostatic indexes may be determined based on a population distribution or over varying points of time. The following table illustrates mechanisms for calculating allostatic load index:

| | |
|---|---|
| Group allostatic | Summary measure representing the number of |
| load index | biomarkers falling within a high risk percentile |
| | (i.e., upper or lower 25th percentile) based on the |
| | sample's distribution of biomarker values. Because |
| | each biomarker is dichotomized as 0 or 1 |
| | depending on cut-offs, each biomarker is allotted an |
| | equal weight in the index. This is the traditional |
| | count-based formulation used most often. |
| z-Score | Summary measure representing the number of |
| allostatic load | biomarkers falling within a high risk percentile |
| index | (i.e., upper or lower 25th percentile) based on a |
| | population's distribution of normative biomarker |
| | values used in clinical practice. This count-based |
| | formulation is pending established biomarker |
| | norms and therefore not yet used in the reviewed |
| | studies. |
| Difference | Difference between two time-points for a single |
| allostatic load | biomarker or an index measure of multiple |
| score | biomarkers. For example, an index measure of pro- |
| | coagulation responses using several hemostastic |
| | biomarkers or two measures of cortisol before and |
| | after exposure to an acute stressor. |
| Dynamic | Repeated measures analysis or change scores |
| allostatic load | between three or more time-points for single or |
| score | multiple biomarkers. For example, diurnal |
| | measures of cortisol at different times of the day |
| | or an area under the curve calculation that |
| | encompasses variability in cardiovascular |
| | functioning over time. |
| Nominal | Dividing participants into groups based on an |
| allostatic load | allostatic load index threshold (e.g. <=3or>=4). |
| grouping | The threshold cut-point can be based on previous |
| | studies with similar number of biomarkers or |
| | arbitrarily based on the sample's distribution. |
| Bootstrapping | Resampling technique used to make inferences |
| | about population parameters by generating multiple |
| | repetitive computations that estimate the shape of a |
| | statistic's sampling distribution (Mooney and Duval, |
| | 1993). The obtained bootstrap statistic can then |
| | be used as weights for allostatic load biomarkers |
| | and/or indices in subsequent analyses. |
| Canonical | Multiple correlational analysis that measures the |
| correlation | association between two sets of latent variables |
| | representing an independent set and a dependent |
| | set. It has been used to determine the best linear |
| | combinations of weighted allostatic load biomarkers |
| | at baseline that are maximally correlated to tertiary |
| | outcomes like mortality at follow-up. |
| Recursive | Multivariate reduction technique that generates |
| partitioning | categories aimed at precisely classifying |
| | participants based on several dichotomous |
| | dependent variables. It has been used to classify |
| | participants into outcome risk categories by first |
| | identifying the biological markers and cut-points |
| | that best differentiate across participants. These |
| | have been used to define allostatic load categories |
| | (e.g., high, intermediate, low) and tertiary outcomes |
| | (e.g., mortality). |
| Grade of | Multivariate reduction technique that identifies |
| membership | heterogeneous groups of combinations and their |
| | value zones that is then used to estimate whether a |
| | participant matches a defined combination, as well |
| | as the degree of their membership into one of |
| | these combinations. A set of individualized |
| | weights is then used to compare participants against |
| | certain pre-defined profiles (e.g., low neuroendocrine |
| | and high metabolic combinations versus high |
| | neuroendocrine and high cardiovascular, etc.). |
| k-Means cluster | Multivariate reduction technique that identifies |
| analysis | homogeneous groups of cases that are then sorted |
| | into one of any specified number of clusters. |
| | Once sorted using a nearest centroid algorithm, |
| | these clusters serve as groups (e.g., recovered, |
| | non-recovered, and fatigued) that can then |
| | compared in terms of allostatic load levels. |
| Genetic | Regression and classification technique involving |
| programming | an evolutionary computer simulation that processes |
| based symbolic | programs built from specified primitives (logical |
| regression | or arithmetic operators such as “+, *, /”) that are a |
| algorithms | good fit to a given dataset. This is a computer |
| | intensive approach ultimately used to understand |
| | the dependency of one variable on several others |
| | (e.g., allostatic load biomarkers and chronic |
| | fatigue syndrome symptoms). |

By way of example, the system may apply the “Group Allostatic Load Index” for stress assessment (302 or 306). A sample group of morning cortisol values may be measured as between 5-23 micrograms per deciliter (mcg/dL) (or 138-635 nanomoles per liter (nmol/L)), whereby 25% of a sample population measured above 20 micrograms per deciliter. If a user is measured in the morning as having cortisol levels of 21 micrograms per deciliter, the allostatic load index for the cortisol biomarker of that user would be dichotomized as a 1. If the remaining measured biomarkers, such as dopamine and epinephrine, are found to be within the middle 50^{th} percentile of a sample population, the allostatic load index for these biomarkers would be each dichotomized as 0. Hence, the user's final allostatic load index for that period of time would calculate to 1.

Allostatic load index may then be plotted on a scheduled, such as weekly, basis. For example, as measurements are taken once per week or the average of more than one measurement per week. Measurements may be made across a predetermined period, such as 90 days, and used to make either an initial stress assessment or subsequent stress assessment. Hence a user who has an average allostatic load index of 5 across several weeks, the value of 5 may be used as a key value for stress assessment. One example of allostatic load data plotted in such a fashion is shown in FIG. 9a.

Alternatively or in addition to the mini-lab 404 performing the above analyses, the home health hub 407 may act as a conduit to transmit to the raw bio-data through network 402 for processing to be performed remotely to determine the biomarker data constituency. The mini-biolab may transmit the data to cloud via smartphone or other internet routing device or hotspot. The current system also contemplates transmitting data that has been analyzed to the cloud, where a health care professional or stress-management expert may review the computer-analyzed results to provide coaching, feedback, prescriptions or other interventions.

Similar assessments (steps 302 and 306 of FIG. 3) may be conducted for physiological and psychological stress data. As described above, the physiological apparatus, such as the wearable patch (405), may measure any number of biosensing indicators such as galvanic skin response, breathing rate or blood oxygen levels as an indications of stress. HRV, as an illustrative measurement used in stress assessments 302 or 306, may be determined through data acquired from wearable patch (405) using a number of techniques known to those of ordinary skill in the art, including time domain techniques, frequency domain techniques, geometric techniques, non-linear method techniques and long-term correlation techniques. Time domain techniques include, but are not limited to: SDNN (statistical deviation of NN (beat-to-beat) intervals), RMSSD (root mean square of successive differences in NN intervals), SDSD (standard deviation of successive differences in NN intervals) and more. Geometric techniques include converting NN intervals into geometric patterns and identify the geometric property variability of the patterns (such as Lorenz plots, for example). Those having ordinary skill in the art will recognize the application of HRV analysis techniques that can be applied to data obtained from wearable patch (405).

By way of example, a user's baseline HRV using an SDNN technique may be determined to be 40 ms, whereas a predetermined lower bound value for a healthy HRV using a SDNN technique may be 50 ms. Hence, the system may determine the user is carrying unhealthy levels of stress. The stress index may be quantified as a percentage deviation from the lower bound, which in this example would be 20%=(50 ms−40 ms)/50 ms). The lower bound value for determining a healthy HRV may be based on a sample of the general population, or stratified by gender, age, ethnicity, etc. The system may also establish a user's baseline HRV during baseline measurement (300), and calculate drops from the percentage baseline rather than from an external measure taken from the general population or some subset thereof. The system may normalize the percentage (e.g., 20% is normalized to 2) for use alone in stress assessments (302 or 306) and/or may apply the value (normalized or not) as input into a formula considering other measured biometrics such as allostatic load (described above) or PSS (described below)

As described above, input from survey answers to a psychometric assessment may be used in stress assessment 302 or 306. The perceived stress scale or other means for gathering psychometric data may be used to track stress on an hourly, daily, weekly, or monthly basis for a predetermined period used to assess stress, such as 90 days. Such input may be gathered during baseline measurement step 300 or some point after in the process of FIG. 3. As an illustrative example, FIG. 9c shows a tracking of perceived stress scale measurements (with a scale of 40 that is obtained by reversing responses (e.g., 0=4, 1=3, 2=2, 3=1 & 4=0) to the four positively stated items (questions 4, 5, 7, & 8) (presented above) and then summing across all questions (also presented above)).

By way of example, a user may provide the following responses set to questions 1-10 respectively of the PSS questionnaire shown above: (2, 3, 1, 3, 3, 0, 3, 3, 2, 1). By reversing responses to questions 4, 5, 7 & 8 and tallying the values, the user receives a psychological assessment rating of 13. This value may be directly used by itself in steps 302 or 306 to assess the stress of an individual, for example by comparing the value to a predetermined threshold level of psychological stress deemed too high, for example a PSS level of 30. Thus, the user in the above example would not be deemed to have a high psychological stress levels. In other embodiments, the PSS stress levels may be input into a function to be combined with other stress assessment levels (e.g., biological or physiological). In such instances, the PSS assessment value may be divided 10, such that the user receiving an assessment of 13 in the example above is given a value of 1.3 for normalization before being input into a function that also takes as inputs allostatic load index and physiological stress level inputs.

The data obtained from the physiological sensors (e.g., wearable patch), biological sensors (e.g., mini-lab) and psychological state surveys (e.g., perceived stress scale data) may be correlated at various points of time. A weighted formula with each components may be applied to identify whether stress levels are improving within a predetermined period. Time-based correlations amongst either the individual data components or aggregated data may be used to assess stress levels at particular points in time and whether stress is improving or deteriorating over time as described in items 302, 306 and 308 of FIG. 3. Further, individual components may indicate a particular area of stress that can be the basis of identifying and recommending a stress interventions, as also described above with respect to process of FIG. 3.

FIGS. 9a, 9b and 9c are graphs illustrating each of three types of measured stress levels, samples once per week, over a period of 90 days. FIG. 9c illustrates psychological assessment levels taking from PSS survey responses and divided by a factor 10 (for normalization and combining with other measurement types). FIG. 9a illustrates weekly allostatic load assessments using a group allostatic load index, as described above. FIG. 9b illustrates physiological assessments made by tracking then number of average hours per night a user deviated from a predetermined target sleep amount of 8 hours. In one embodiment, any one of these types of measured stress levels are used directly as the stress level assessment. In another embodiment, each of type of measured stress levels are assessed, and the lowest normalized assessment is the basis of stress intervention and/or goal adoption 304. Continuing this example, the physiological stress index during week 1, having a value of “3” is the lowest normalized assessment when compared to the PSS and allostatic load index, and therefore would be the basis of stress intervention and/or goal adoption In yet another embodiment, each of the three types of assessments are put into a function, the output of which is compared to a predetermined value. In a simple example, each of the weekly PSS index, Allostatic load index, and Physiological stress index values are added and compared to a predetermined value. Thus, for assessing the stress levels of week 1, the 3.5 (the PSS level) would be added to 6 (the allostatic load index) and to 3 (the physiological load index) to yield an overall stress assessment level of 12.5.

For example, if a user's physiological systems indicate a low resting heart rate (and therefore healthy physical conditioning), but allostatic load (as measured through cortisol levels) and perceived stress (as measured through PSS indicate) suggest high levels of stress, a meditation intervention rather than exercise module may be suggested. Analysis of correlated time stamp data may be the basis for any number of interventions/models such as quitting smoking, improving sleep, etc. . . . .

In another embodiment, the system, apparatus and method may be applicable for managing the health of animals.

It should be understood that the descriptions included here of logic and logical statements may be implemented through hardware, firmware (or other such low-level software), programmatic and updatable applications (or other such high-level software) and/or a combination thereof. Hardware implementations disclosed may include various electronic circuitry, microprocessors, signal processors, controllers, etc. Software implementations disclosed herein may include source code that is compiled directly or higher-order programmatic schemes that may include calls and functions in reference to hardware components or basic action-functions.

Aspects of teachings provided such as the logical flows may include signals that may be read and/or stored on various types of media including hardware or non-transitory computer-readable media, such as a floppy disk drive, Compact Disc (CD), Universal Serial Bus (USB) drive, computer memory devices (read only memory (ROM), random access memory (RAM), flash, etc.)

Logical flows and operations performed, as described in the instant specification, should not be understood to be limited by the order in which they are presented. Some embodiments may be order-independent, and thus there may be several permutations in which the stages making up an operational flow may be carried out. In some instances, stages may be carried out concurrently, and in distinct computing and geographic environments.

All definitions, as defined and used herein, should be understood to control over dictionary definitions, definitions in documents incorporated by reference, and/or ordinary meanings of the defined terms.

The indefinite articles “a” and “an,” as used herein, unless clearly indicated to the contrary, should be understood to mean “at least one.”

As used herein, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “and/or,” as used herein, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items.

The phraseology and terminology used herein is for the purpose of description and should not be regarded as limiting. The use of “including,” “comprising,” “having,” “containing”, “involving”, and variations thereof, is meant to encompass the items listed thereafter and additional items.

Having described several embodiments of the disclosure in detail, various modifications, and improvements will readily occur to those skilled in the art. Such modifications and improvements are intended to be within the spirit and scope of the disclosure. Accordingly, the foregoing description is by way of example only, and is not intended as limiting.

Several references to the figures of this patent application are made in this Detailed Description. Illustrative embodiments disclosed herein are intended to provide sufficient written description, enable one having ordinary skill in the art to make and/or use the claimed disclosure without undue experimentation and otherwise meet the requirements of 35 U.S.C. §112. Such disclosure is not meant to be limiting, as other embodiments with adaptations may be employed without departing from the spirit or scope of the subject matter presented herein.

1. A system for individualized stress management comprising:
a processing unit; and
a set of user-interfacing devices for collecting user data, the user data including physiological data and biological data derived from a saliva sample;
wherein the set of user interfacing devices are connected to the processing unit to transmit the collected user data for determination of individualized stress management by coordinating heart rate or heart rate variability determined from the physiological data and determining an allostatic load index by plotting cortisol density measurements from the user's saliva samples within a distribution of a population's cortisol density measurement.

2. The system of claim 1 wherein the set of user-interfacing devices comprises:
at least one physiological data device configured to measure physiological data; and
at least one biological data device configured to measure allostatic load data.

3. The system of claim 2 wherein the set of user-interfacing devices further comprises:
at least one psychological data device for collecting user data in the form of psychological data.

4. (canceled)

5. The system of claim 2 wherein the at least one physiological data device comprises:
a wearable patch with a set of electrodes integrated within the wearable patch.

6. The system of claim 5 wherein the set of electrodes comprises at least one of an electro-cardiogram (ECG) sensor, an inertial measurement unit (IMU), a thermistor, a microphone, a pulse-oximetry sensor, a proximity sensor and a global positioning (GPS) sensor.

7. The system of claim 5 wherein the at least one physiological device comprises a electroencephalogram (EEG) sensors, an electrooculography (EOG) sensor, a glucose sensor, a breath analyzer monitors, a blood pressure monitor, a plethysmograph, a respiration sensors, an electro-dermal sensor or a fluid detector.

8. A method of stress management comprising:
performing an initial stress assessment based on a set of initial stress assessment measurements; and
recommending a stress reduction intervention program based on the initial stress assessment by comparing the set of initial stress assessment measurements with a set of baseline measurements;
wherein each of the set of initial stress assessment measurements and set of baseline measurements include biological data and physiological data, the biological data including allostatic load data.

9. The method of claim 8 further comprising:
performing a subsequent stress assessment based on a set of subsequent stress assessment measurements; and
comparing the subsequent stress assessment with a previous stress assessment.

10. The method of claim 9 further comprising:
modifying the stress intervention program based on the comparison between the subsequent stress assessment and the previous stress assessment.

11-13. (canceled)

14. The system of claim 1 wherein the allostatic load data comprises neuroendocrine, immune, metabolic, cardiovascular or anthropometric biomarker data.

15. The system of claim 1 further comprising a psychometric diagnostic apparatus.

16. The system of claim 1 wherein the processing unit comprises:
a physiological apparatus interaction module;
a stress intervention module; and
a biological data apparatus interaction module.

17. The system of claim 16 wherein the stress intervention module determines an individualized stress management plan.

18. The system of claim 16 wherein the biological data apparatus interaction module receives data from the biological data apparatus.

19. The system of claim 16 further comprising a psychological apparatus interaction module.

20. The system of claim 1 wherein the processing unit transmits and receives data from a server.

21. The system of claim 5 wherein the patch is flexible.

22. The system of claim 5 wherein the wearable patch comprises a disposable patch.

23. The system of claim 22 wherein the disposable patch is an adhesive hydrogel layer.
